(19) United States
(12) Patent Application Publication
(10) Pub. No.: US 2006/0060204 A1
(43) Pub. Date: Mar. 23, 2006

(54) Double dosimetric dropper assembly for nasal or similar application

(76) Inventor: Regina Maria Fuentes, Sao Paulo (BR)

Correspondence Address: D. PETER HOCHBERG CO. L.P.A., 1940 EAST 6TH STREET, CLEVELAND, OH, 44114, US

(21) Appl. No.: 11/229,820

(22) Filed: Sep. 19, 2005

(30) Foreign Application Priority Data
Sep. 20, 2004 (BR) MU 8402407-0

(51) Int. Cl. A61M 15/08; A62B 7/00

(52) U.S. Cl. 128/207.18

(57) ABSTRACT

A double dosimetric dropper assembly for nasal or similar application comprises a nasal medicine liquid dosage and application system having a threadable lid with a double dropper mounted to the lid in a concentric position, an elastomeric syringe bulb and a pitch-shaped double capillary. The syringe bulb works as an hydraulic pump which, after being finger-squeezed, returns to its original position, withdrawing by suction the liquid medicine in a correct dosage by the double capillary's action. The capillary tubes are installed in their lower terminal for drawing the bottle's liquid to its inner part; when the syringe bulb is compressed again, it expels the same liquid into the nostrils. The dosimetric double dropper has two pitch-shaped capillaries for nasal medicine application into the two nostrils at the same time and with equal dosage in a single operation.

### FIELD OF THE INVENTION

The present invention relates generally to the packaging field and use, and more specifically to pharmaceutical bottles and containers, for antiseptic products, medicines and specific serums administered simultaneously into two nostrils.

### DESCRIPTION OF THE PRIOR ART

The bottle-and-container-shaped packages are widely used for storing medical liquid products. They are the preferred containers in the pharmaceutical industry, avoiding unwanted losses of content, in addition to being easily handled by the user.

Similarly, the nozzles with lids and dosemeters are often used in containers in the pharmaceutical industry since they allow medicine liquids to be purchased and easily used by the users.

In the process of preparing the lids for the receptacles and containers for medical use, initially, droppers were inserted from the upper part of the containers' lids for attachment to the glass nozzle, the nozzle containing small, elastomeric-material suction pumps common at that time. A dropper would also have a glass capillary concentrically introduced in its lower part which would draw the medicine using suction, to the user. Such disposition has shown to be efficient for sealing the container, and for preservation and dosage of its content. However, experience has shown that such disposition has limitations in relation to the access to the container's or receptacle's content, since the user will always need to make an effort to open the container owing to existence of fitted lids, often resulting in spilling of the container's content.

Another bothersome and important problem of the prior art was because the dropper's components were manufactured with glass material and with insecure mounting techniques of its mechanical elements, which gave rise to accidents from breaking and contamination due to missealing of the containers.

Owing to these drawbacks and to the adopted practices, many constructive dispositions have been developed, such as the threadable lid system and droppers externally provided with elastomeric pumps or suction valves, which have improved the drainage, sealing, service and asepsis condition of the dropper, in addition to their being easily opened and/or closed.

However, in addition to the existing drawbacks and the ones which have appeared, it has been observed that together with the new technologies applied to the nasal medicines, comes the need of a better qualitative and quantitative control regarding the dosage which the patient and user should receive owing to the different concentrations of active principles of specific medicines, where the existing application devices no longer offer an efficient support to what would be reasonable.

It is observed, owing to the practical facts presented, that the presence of a single dosimetric capillary of service and suction can no longer assure the dosage efficiency and the exact balance control in correlation to how much medicine should be applied at the same time to the two nostrils in the same operation.

Aiming at the improvement of the droppers' operational attributes, herein is presented a practical and innovating model of threadable lid system and dropper with all aesthetical and functional qualities, designed and developed in compliance with the most modern techniques, enabling this way its most different use, in bottles provided with a bottleneck for the storage of liquid medicines.

The present invention's innovating concept allows for the obtaining of an excellent level of operation, offering a lid system model with a double dropper, which functions well and is provided to make applying medical therapy liquids easier for the user, and, at the same time, to guarantee providing the necessary dosage, lessening the errors owing to manipulation.

An object of the present patent is the provision of a threadable lid system with a double dropper which allows its threading to the container's nozzle profile.

The device according to a preferred embodiment of the invention, whose application and dosage system of nasal liquid medicines is characterized by a lid with a double dropper, wherein one threadable lid is coupled in a concentric position with an elastomeric suction device and a pitch-shaped double capillary, the capillary being built from engineering material such as high-strength transparent plastic. The suction device with the tubular shape of closed cylindrical top works as an hydraulic pump which, after being finger-squeezed, returns to its original position, drawing by suction the correct liquid medicine dose, which by the action of the double capillary installed in its lower terminal, draws the liquid from the bottle to the inside of the suction device and when compressed again, expels the same liquid into the nostrils.

The dosimetric double dropper has the innovation of two pitch-shaped capillaries or capillary tubes for application of nasal medicine into the two nostrils at the same time and with equal dosage in a single operation.

The suction device is coupled efficiently by the interference of a concentric hole of the container's lid, preventing spillage and contamination of its medicine content and helps the liquid drainage flux control from the inner receptacle to the external means by the dropper double capillary. The double capillary acts efficiently, controlling that flow, complemented by the threadable lid which hinders the flow from the container completely, even with the receptacle upside-down.

The object of the present invention, in addition to constructiveness, functionality and simplicity, is also to provide a device with hygienic properties such as the asepsis in service, improving its commercial value and the user's satisfaction.

Therefore, the present invention incorporates not only the mechanical and functional qualities that have been taken into account in its manufacture, but also the form, the disposition and the location of its parts and components which, correctly dimensioned and built with modern materials, have provided an improvement without increasing its price in any way.

Thus, the present invention has been designed with the goal of obtaining a device with the least possible number of parts, conveniently set up and displayed so as to perform its functions with high efficiency and versatility, without the already mentioned drawbacks of the prior art.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 shows a front view of the lid with double dropper for nasal application;

FIG. 2 shows a top view of the lid with double dropper for nasal application;

FIG. 3 shows a top-frontal perspective view of the lid with double dropper for nasal application;

FIG. 4 shows a bottom view of the lid with double dropper for nasal application;

FIG. 5 shows a bottom-frontal perspective view of the lid with double dropper for nasal application;

FIG. 6 shows an exploded top-frontal perspective view of the lid with double dropper;

FIG. 7 shows a top-frontal perspective cut view of the lid with double dropper for nasal application;

FIG. 8 shows a view of the lid with double dropper for nasal application being handled; and

FIG. 9 shows a top-frontal perspective view of the lid with double dropper for nasal application with a bottle, which receives it.

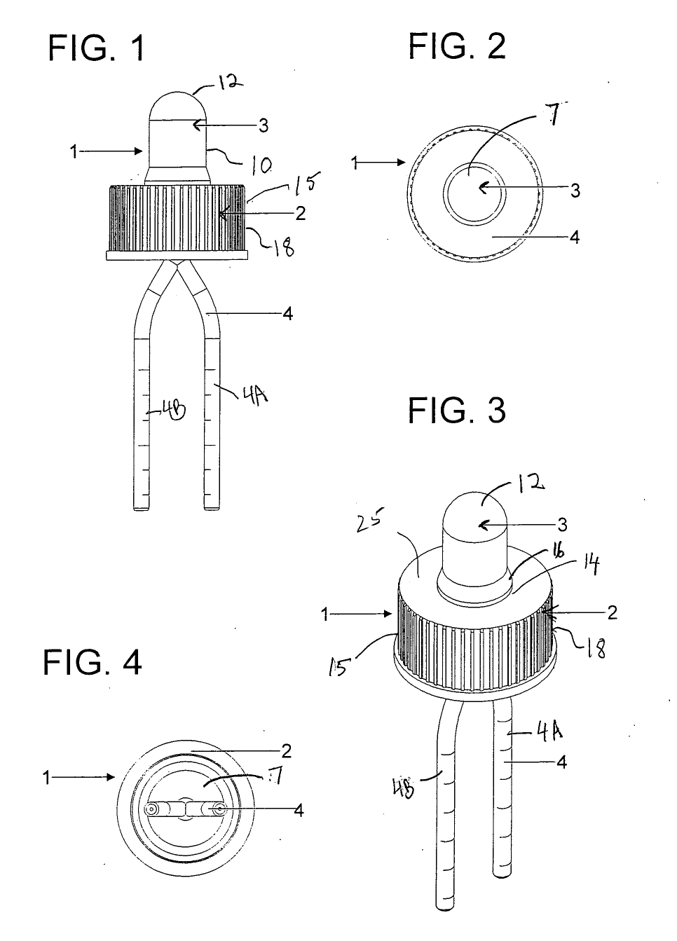

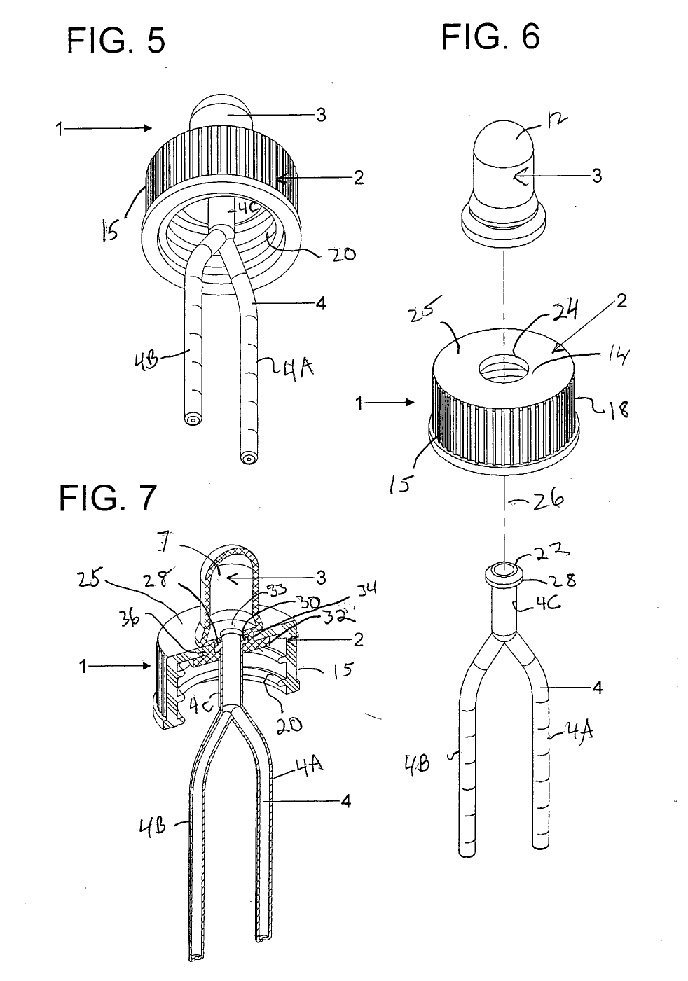

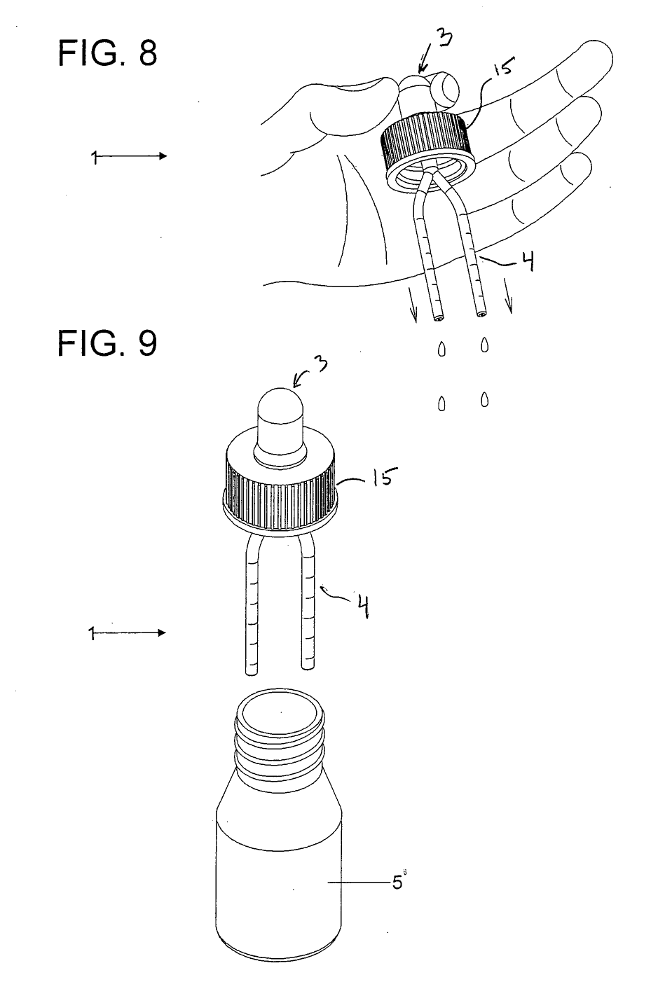

### DETAILED DESCRIPTION OF THE PREFERRED EMBODIMENT

A double dosimetric dropper for nasal or similar application according to a preferred embodiment of the invention constitutes an application and dosage system or double dosimetric dropper assembly 1 of nasal liquid medicine, characterized by a lid with double dropper 4, wherein a threadable lid 2 is coupled in concentric position to an elastomeric suction device or syringe bulb 3 and to the double dropper or pitch-shaped double capillary 4. The double capillary is built with engineering material such as a hard, transparent plastic which is relatively strong and rigid. The syringe bulb 3 with a closed, hemispherical top 12 having a cylindrical, tubulat-shaped configuration works as an hydraulic pump which, after being finger-squeezed, returns to its original position since it is made from a resilient elastomeric material. Syringe bulb 3 withdraws under suction the bottle's liquid medicine to the inside 7 of bulb 3 and when compressed again, expels the same liquid into the nostrils.

The dosimetric double dropper assembly 1 has the innovation of two pitch-shaped capillaries 4 for nasal medicine application into the two nostrils at the same time and with equal dosage in a single operation.

Considering dosimetric double dropper assembly 1 in further detail, syringe bulb 3 comprises a cylindrical body 10 having hemispherical top 12 and an annular groove (discussed below) for receiving an inner rim 14 of lid 2. The lower portion of syringe bulb 3 has a flared-out portion 16 for helping to define the groove.

Lid 2 has a cylindrical rim 15 with longitudinal, axial ridges and grooves 18 for providing friction to enable double dropper assembly 1 to be screwed and unscrewed from a bottle or other container 5. Lid 2 further has internal threads 20 on the inside of rim 15 for being screwed and unscrewed from a correspondingly threaded container. Lid 2 is preferably made from a hard plastic or possibly a metal.

Double capillary 4 is composed of a pair of capillaries or capillary tubes 4A and 4B which each extend from a central tube 4C having an open end 22. The upper portion of central tube 4C extends to a central hole 24 in the center of a top 25 of lid 2 along a longitudinal axis 26 which extends along the center of syringe bulb 3. Central tube 4C further has an annular flange 28 which fits into an inner annular recess 30 in syringe bulb 3 to provide a liquid seal between flange 28 of tube 4C and bulb 3.

With particular reference to FIG. 7, it can be seen that syringe bulb 3 further has a laterally extending lower base 32, a parallel upper base 33 and an annular section 34 for defining both inner annular recess 30 and an outer annular recess 36 which engages inner rim 14 defining hole 24 to establish a fluid seal between syringe bulb 3 and lid 2. Annular recesses 30 and 36 are further defined by an annular wall 38 coaxial with axis 26, which defines the depth of both recesses.

As explained earlier, when double dropper assembly 1 is in a container holding a liquid medicine, the syringe bulb 3 is squeezed and released to draw liquid medicine through tubes 4A and 4B through central tube 4C and into bulb 3. Thereafter, tubes 4A and 4B are inserted into nostrils, and bulb 3 is squeezed to eject the liquid medicine in a controlled and accurate manner.

The lid with double dosimetric dropper for nasal or similar application according to the invention is a device of great use for withdrawing suction liquid medicine under a controlled dosage, where the lid seals the container and provides a hygienic device.

The invention has been described with particular emphasis on the preferred embodiments. It should be appreciated that these embodiments are described for purposes of illustration only, and that numerous alterations and modifications may be practiced by those skilled in the art without departing from the spirit and scope of the invention. It is intended that all such modifications and alterations be included insofar as they come within the scope of the invention or the equivalents thereof.

What is claimed is:

1. A device for a dosage system of liquid nasal medicine, having a bottle fillable with liquid nasal medicine, said device comprising:
a lid threadable for mating with the bottle;
a double dosimetric dropper having two pitch-shaped capillaries connected to a tube attached to one side of the lid; and
an elastomeric suction device attached to the lid on the opposite side from the lower branch of the dropper;
wherein a first squeezing of said suction device sucks a correct dosage of said liquid nasal medicine into said capillaries and a second squeezing expels the correct dosage of the liquid.

2. A device according to claim 1, wherein said correct dosage is the same amount of liquid medicine in each capillary.

3. A device according to claim 1 wherein said suction device is a syringe bulb.

4. A double dosimetric dropper assembly comprising:
a lid for being screwed onto a threaded container, said lid including:
a cylindrical rim defining a longitudinal axis and ridges and grooves for providing a surface to be gripped by a person's hand for screwing or unscrewing said lid from a container; and
a top extending across said rim and having a concentric hole defined by an inner rim;
an elastomeric syringe bulb including:
a squeezable portion for being disposed on said top and out of a container for holding and discharging a liquid;
an upper base extending across said squeezable portion for engagement with the outside of said lid and having an axial hole for alignment with the hole in said lid;
a lower base parallel with said upper base for engagement with the inside of said lid, said lower base having an axial hole for alignment with the hole in said lid; and
an annular wall extending between said upper base and said lower base to define an outer annular recess for receiving said inner rim of said lid and an inner recess; and
a double dosimetric dropper comprising:
a central tube having an opening for alignment with said opening in said lid, and a flange for being received in a fluid-tight manner in said inner recess of said syringe bulb; and
a pair of capillaries extending from said central tube, each capillary having a liquid-carrying portion for carrying liquid from and to said central tube, said pair of capillaries having parallel free end portions;
said squeezable portion of said syringe bulb being squeezable to exert suction on liquid in a container to withdraw the liquid from the container, through said dosimetric dropper and into said bulb; and said capillaries being thereafter insertable into a pair of nostrils, said bulb being squeezable to discharge liquid from said bulb into the nostrils by an equal dosage into each nostril.
